(19) United States
(12) Patent Application Publication
(10) Pub. No.: US 2021/0113430 A1
(43) Pub. Date: Apr. 22, 2021

(54) SAFETY SHOWERS AND EYE-WASHING APPARATUS

(71) Applicant: Hughes Safety Showers Limited, Bredbury (GB)

(72) Inventors: Keshar Nath, Wilton, CT (US); Alan Ludlaim, Bredbury (GB); Kiran Damji, Bredbury (GB)

(73) Assignee: Hughes Safety Showers Limited, Bredbury (GB)

(21) Appl. No.: 16/874,805

(22) Filed: May 15, 2020

Related U.S. Application Data
(63) Continuation of application No. PCT/US2020/023736, filed on Mar. 19, 2020.

(30) Foreign Application Priority Data
Oct. 17, 2019 (GB) 19150556

(51) Int. Cl. A61H 35/02; G01N 33/18; G08B 21/18; G06K 9/00

(52) U.S. Cl. CPC A61H 35/02; G01N 33/1826; G01S 19/42; G08B 21/18; G06K 9/00228; G01N 33/18

(57) ABSTRACT

An apparatus and method for remote monitoring and data capture in an emergency washing apparatus adapted for generating a controlled spray or jet of water directed at a user for washing therewith. A washing unit includes a water inlet and one or more water outlets arranged for forming a spray or jet of water received from the water inlet. One or more sensors is configured to sense one or more properties of the water received at the water inlet selected from: a water flow rate, a water pressure, a water temperature, a water pH level, a bacteria content. The one or more sensors are configured to generate a representative measurement signal indicating a value of the one or more sensed properties of the water, and to transmit the measurement signal for reception locally at, or remotely from, the washing unit.

### RELATED APPLICATIONS

This application is a continuation of International Application No. PCT/US2020/023736, filed Mar. 19, 2020, which claims the benefit of GB Patent Application No. 1915055.6, filed Oct. 17, 2019, the contents of which are incorporated herein by reference.

### FIELD

The disclosure generally relates to safety shower stations and/or eye washing stations, such as emergency showers and/or eye washing stations, for containing and dispensing a shower, spray or jet of water with which to rinse, bathe or wash a part of a user's body and/or eyes.

### BACKGROUND

In industrial settings, such as in chemical processing plants or the like, personnel may be exposed to potentially harmful chemicals or materials in liquid, gaseous, vapor or powder form from time to time. Should such harmful materials contaminate the skin and/or eyes of a member of personnel, then it is essential that the skin and/or eyes of the member of personnel are rinsed, bathed or washed as soon as possible and as a matter of urgency so as to remove the contaminant materials and thereby limit damage to them.

It is common practice that, in such industrial settings, emergency shower stations and/or eye washing stations are provided at which a supply of washing fluid (typically water) is stored for this use exclusively. Such emergency shower stations and/or eye washing stations may often consist of a water tank or reservoir to which is connected a water dispensing shower head or nozzle adapted for providing an output spray or jet of water to be directed over the user, or into the eyes of the user, for washing.

Given that long periods of time may pass between successive uses of these emergency shower/washing stations, un-used water is often found to accumulate and stagnate within the water tanks and/or water conduits of the apparatus between uses. This means that, any successive use of the shower/washing station will cause the eyes of the user to be based, at least initially, with stagnant water remaining from the previous use of the apparatus. This stagnant water is likely to contain harmful bacteria which could be very damaging to the health of the user. A further problem that can arise from long periods of inactivity is that the water flow control valves of a shower may seize up due to corrosion or an accumulation of debris in between moving parts of the valve mechanism, over time, which impedes or prevents their proper movement.

In addition, in order to be effective it is necessary that a suitable rate of flow of water is output from the water outlet (e.g. shower head or eye-wash nozzle) sufficient to permit effective washing of the user's skin and/or eyes. Faults in the equipment could cause a user to suffer avoidable harm due to ineffective operation when needed most—e.g. in an emergency. Thus, it can be important that the equipment is regularly checked and tested in relevant ways to ensure that they remain compliant with local, national standards and safety regulations (e.g. European Standard “EN 15154—Safety showers”; “ANSI Z358.1 Safety Shower & Eye Wash Regulations”).

The present invention aims to address these matters.

### SUMMARY

The invention may provide an apparatus and method for remote monitoring and data capture in an emergency washing apparatus.

In a first aspect, the invention may provide an emergency washing apparatus adapted for generating a controlled spray or jet of water directed at a user for washing therewith, the apparatus comprising: a washing unit including a water inlet and one or more water outlets arranged for forming the spray or jet of water received from the water inlet; one or more sensors configured to sense one or more properties of the water received at the water inlet selected from: a water flow rate; a water pressure; a water temperature; a water pH level, a bacteria content; wherein the one or more sensors are configured to generate a respective measurement signal indicating a value of the one or more sensed properties of the water, and to transmit the measurement signal for reception locally at, or remotely from, the washing unit.

The emergency washing system may include a shower control unit which is located locally at, or remote from, the washing unit and which is arranged to receive the respective measurement signals and to issue a warning signal if the received measurement signals fail to comply with respective pre-set conditions thereby indicating a malfunction of the washing unit.

The emergency washing system may include one or more remotely controllable water flow control valves each configured in fluid communication with the water inlet and with a respective one of the one or more water outlets, wherein each remotely controllable water flow control valve is operable to reversibly place a respective said water outlet in fluid communication with the water inlet. The shower control unit is preferably arranged to remotely control one or more of the remotely controllable water flow control valves to reversibly place a respective said water outlet in fluid communication with the water inlet.

The shower control unit may be arranged to issue a said warning signal if a plurality of the received measurement signals fail to comply concurrently with pre-set conditions whereby concurrent compliance with the pre-set conditions is such that a condition applied to one of the received measurement signals is conditional upon another said received monitoring signal.

The emergency washing system may include a shower control unit which is located locally at, or remote from, the washing unit and which is arranged to receive the respective measurement signals, wherein the shower control unit includes a data processor and a display unit configured to generate and display a graphical user interface displaying one or more of the sensed properties of the water.

The data processor and display unit are preferably configured to issue the warning signal as a warning displayed by the graphical user interface.

The washing unit may include a user identity unit configured to generate identification information identifying a user of the washing unit and to transmit the user identification information. Desirably, the emergency washing system includes a shower control unit located locally at, or remote from the washing unit and responsive to receipt of the transmitted user identification information to issue a telecommunications signal conveying information selected from: information identifying the location of the washing unit; and, information identifying the user.

The data processor and display unit may be configured to generate one or each of: geographical coordinates identifying the location of the washing unit; a map of an environment local to the washing unit upon which the location of the washing unit is shown, and to display the geographical coordinates or map on the graphical user interface. For example, the geographical coordinates may then be used to show the location of the washing unit on an online mapping application/software (e.g. Google Maps).

In a corresponding aspect, the invention may provide an emergency washing method for in an apparatus adapted for generating a controlled spray or jet of water directed at a user for washing therewith, the method comprising: providing a washing unit including a water inlet and one or more water outlets and therewith forming the spray or jet of water received from the water inlet; providing one or more sensors and therewith sensing one or more properties of the water received at the water inlet selected from: a water flow rate; a water pressure; a water temperature; a water pH level, a bacteria content;

by the one or more sensors, generating a respective measurement signal indicating a value of the one or more sensed properties of the water, and transmitting the measurement signal for reception locally at, or remotely from, the washing unit.

The method may include providing a shower control unit which is located locally at, or remote from, the washing unit and therewith receiving the respective measurement signals and issuing a warning signal if the received measurement signals fail to comply with respective pre-set conditions thereby indicating a malfunction of the washing unit.

The method may include providing one or more remotely controllable water flow control valves each configured in fluid communication with the water inlet and with a respective one of the one or more water outlets, and remotely controlling one or more water flow control valves to reversibly place a respective water outlet in fluid communication with the water inlet. The shower control unit is preferably arranged to remotely control the one or more of the remotely controllable water flow control valves in this way.

The method may include, by the shower control unit, issuing a said warning signal if a plurality of the received measurement signals fail to comply concurrently with pre-set conditions whereby concurrent compliance with the pre-set conditions is such that a condition applied to one the received measurement signals is conditional upon another said received monitoring signal.

The method may include, providing a shower control unit which is located locally at, or remote, from the washing unit and therewith receiving the respective measurement signals and, by the shower control unit, generating and displaying a graphical user interface showing one or more of the sensed properties of the water.

The method may include issuing the warning signal as a warning displayed by the graphical user interface.

The method may include providing a user identity unit and therewith generating identification information identifying a user of the washing unit, and transmitting the user identification information, wherein the method includes providing a shower control unit located locally at, or remote from, the washing unit and therewith responding to receipt of the transmitted user identification information by issuing a telecommunications signal conveying: information identifying the location of the washing unit; and, information identifying the user.

The method may include, by the data processor, generating one or each of:

geographical coordinates identifying the location of the washing unit; a map of an environment local to the washing unit upon which the location of the washing unit is shown, and displaying the geographical coordinates or map on the graphical user interface. For example, the geographical coordinates may then be used to show the location of the washing unit on an online mapping application/software (e.g. Google Maps).

The invention may provide an apparatus and method for remote activation of an emergency washing apparatus.

In a second aspect, the invention may provide an emergency washing apparatus adapted for generating a controlled spray or jet of water directed at a user for washing therewith, the apparatus comprising: a washing unit including a water inlet and one or more water outlets arranged for forming the spray or jet of water received from the water inlet; one or more remotely controllable water flow control valves each configured in fluid communication with the water inlet and with a respective one of the one or more water outlets, wherein each remotely controllable water flow control valve is operable to reversibly place a respective the water outlet in fluid communication with the water inlet; a shower control unit which is remote from the washing unit and which is arranged to remotely control the one or more remotely controllable water flow control valves to reversibly place a respective said water outlet in fluid communication with the water inlet.

The emergency washing apparatus may include a water flow-rate sensor arranged to measure a rate of flow of water from the water inlet to the one or more water outlets, and to generate a monitoring signal indicating a measured rate of flow. Desirably, the shower control unit is arranged to perform remote data gathering by: controlling at least one said remotely controllable water flow control valve to place a respective said water outlet in fluid communication with the water inlet for a finite pre-set time period; and, receiving the monitoring signal during the time period.

The shower control unit may be arranged to issue a warning signal if the measured rate of flow falls below a pre-set threshold water flow rate value.

The shower control unit may be arranged to repeat the remote data gathering if an interval of time since the most recent previous said remote data gathering exceeds a threshold time interval value.

In a corresponding aspect, the invention may provide an emergency washing method for an apparatus adapted for generating a controlled spray or jet of water directed at a user for washing therewith, the method comprising: providing a washing unit including a water inlet and one or more water outlets arranged for forming the spray or jet of water received from the water inlet; providing one or more remotely controllable water flow control valves each configured in fluid communication with the water inlet and with a respective one of the one or more water outlets; providing a shower control unit which is remote from the washing unit and therewith remotely controlling the one or more remotely controllable water flow control valves to reversibly place a respective said water outlet in fluid communication with the water inlet.

The method may include providing a water flow-rate sensor and therewith measuring a rate of flow of water from the water inlet to the one or more water outlets, and generating a monitoring signal indicating a measured rate of flow.

Desirably, the method includes, by the shower control unit, performing remote data gathering by: controlling at least one said remotely controllable water flow control valve to place a respective said water outlet in fluid communication with the water inlet for a finite pre-set time period; and, receiving the monitoring signal during the time period.

The method may include, by the shower control unit, issuing a warning signal if the measured rate of flow falls below a pre-set threshold water flow rate value.

The method may include, by the shower control unit, repeating the remote data gathering if an interval of time since the most recent previous said remote data gathering exceeds a threshold time interval value.

The invention may provide an apparatus and method for remote testing of an emergency washing apparatus according to multiple concurrent test criteria.

The invention may provide an apparatus and method for remote testing of an emergency washing apparatus according to multiple concurrent test criteria.

In a third aspect, the invention may provide an emergency washing apparatus adapted for generating a controlled spray or jet of water directed at a user for washing therewith, the apparatus comprising: a washing unit including a water inlet and one or more water outlets arranged for forming the spray or jet of water received from the water inlet; one or more water flow control valves each operably coupled to a respective control switch arranged to operate the water flow control valve to reversibly place a said water outlet in fluid communication with the water inlet; one or more sensors configured to detect an operation of a said control switch and to transmit a monitoring signal indicating an occurrence of such operation; one or more sensors configured to sense one or more properties of the water received at the water inlet selected from: a water flow rate; a water pressure; a water temperature; and to transmit a respective measurement signal indicating a sensed property of the water; a shower control unit which is located locally at, or remote from, the washing unit and which is arranged to receive the monitoring signals and the measurement signals and to issue a warning signal if the received monitoring signals and measurement signals collectively fail to comply concurrently with pre-set conditions thereby indicating a malfunction of the washing unit.

Preferably, the one or more said control switches are each selected from: manually operable control switches; remotely operable control switches.

Desirably, the concurrent compliance with the pre-set conditions is such that a condition applied to the received measurement signals is conditional upon the received monitoring signals.

The one or more sensors may be configured to sense the water flow rate and the shower control unit may be arranged to issue the warning signal if the measurement signal indicates a non-zero water flow rate and concurrently a said monitoring signal(s) indicates that no said water outlet is in fluid communication with the water inlet.

The one or more sensors may be configured to sense the water flow rate and the shower control unit may be arranged to issue the warning signal if the measurement signal indicates no water flow and concurrently a said monitoring signal(s) indicates that a said water outlet is in fluid communication with the water inlet.

The emergency washing apparatus may comprise a first said water outlet and a second said water outlet, in which the one or more sensors are configured to sense the water flow rate and the shower control unit is arranged to issue the warning signal if the measurement signal indicates a water flow rate exceeding: a first pre-set water flow rate threshold value and concurrently a said monitoring signal(s) indicates that only one of the first and second water outlets is in fluid communication with the water inlet; or, a second pre-set water flow rate threshold value exceeding the first pre-set water flow rate threshold value and concurrently a said monitoring signal(s) indicates that both of the first and second water outlets is in fluid communication with the water inlet.

The first pre-set water flow rate threshold value is preferably between 1 liter per minute and 12 liters per minute, or preferably between 1 liter per minute and 6 liters per minute, or preferably between 6 liters per minute and 12 liters per minute, or preferably be at least 6 liters per minute, or preferably at least 11 liters per minute (e.g. at least 11.4 liters per minute). The first pre-set water flow rate may be applicable to a desired water flow rate for an eye-wash apparatus, and one of the first and second water outlets may comprise an eye-wash nozzle(s). The second pre-set water flow rate threshold value is preferably between 30 liters per minute and 60 liters per minute, or preferably between 60 liters per minute and 100 liters per minute, or may be at least 100 liters per minute. The second pre-set water flow rate may be applicable to a desired water flow rate for a shower head, or for a shower head and an eye-wash apparatus when used concurrently, and one of the first and second water outlets may comprise a shower head, or the first and second water outlets may collectively comprise a shower head and an eye-wash apparatus having an eye-wash nozzle(s).

Accordingly, a lower flow rate is relevant to the use of the eyewash apparatus alone, and the first pre-set water flow rate threshold value may be associated with this. However, a higher flow rate is suitable for the use of the shower alone, while a yet higher flow rate is relevant to the concurrent use of both the eyewash and the shower together, and a respective second pre-set water flow rate threshold value may be selected to be applicable to each of these higher flow rates individually (e.g. a different second pre-set water flow rate threshold values each suitable to one of these two conditions respectively). One or each of the first pre-set water flow rate threshold value and the second pre-set water flow rate threshold value, may be compliant with any one of the following national standards: European Standard “EN 15154—Safety showers”; “ANSI Z358.1 Safety Shower & Eye Wash Regulations”).

The one or more sensors are preferably configured to sense the water pressure and the shower control unit is arranged to issue the warning signal if the measurement signal indicates a water pressure exceeding a pre-set upper pressure threshold value and concurrently a said monitoring signal(s) indicates that no said water outlet is in fluid communication with the water inlet. For example, this could indicate that the water control valve(s) are closed and that the water pressure ‘behind’ them is too high for safe use. For example, formation of a shower or eye-wash water spray of jet using a supply of water at such high pressure may be injurious to the user and/or may damage the equipment of the washing unit.

The one or more sensors are preferably configured to sense the water pressure and the shower control unit is arranged to issue the warning signal if the measurement signal indicates a water pressure less than a pre-set lower pressure threshold value, and concurrently a said monitoring signal(s) indicates that no said water outlet is in fluid communication with the water inlet. For example, this could indicate that the water control valve(s) are open and that the water pressure ‘behind’ them is too low for effective/safe use. For example, formation of a shower or eye-wash water spray of jet using a supply of water at such low pressure may be impossible or ineffective to produce the required spray/jet of water to allow the user to wash effectively.

The pre-set upper pressure threshold value may be between 200 KPa and 600 KPa (e.g. between 2 bar and 6 bar). The pre-set lower pressure threshold value may be between 200 KPa and 600 KPa (e.g. between 2 bar and 6 bar). For example, the pre-set upper pressure threshold value may be 600 KPa, whereas the pre-set lower pressure threshold value may be 200 KPa.

In a corresponding aspect, the invention may provide an emergency washing method adapted for generating a controlled spray or jet of water directed at a user for washing therewith, the method comprising: providing a washing unit including a water inlet and one or more water outlets arranged for forming the spray or jet of water received from the water inlet; providing one or more water flow control valves each operably coupled to a respective control switch arranged to operate the water flow control valve to reversibly place a said water outlet in fluid communication with the water inlet; detecting an operation of a said control switch and transmitting a monitoring signal indicating an occurrence of such operation; sensing one or more properties of the water received at the water inlet selected from: a water flow rate; a water pressure; a water temperature; and to transmit a respective measurement signal indicating a sensed property of the water; providing a shower control unit which is located locally at, or remote from, the washing unit and therewith receiving the monitoring signals and the measurement signals, and issuing a warning signal if the received monitoring signals and measurement signals collectively fail to comply concurrently with pre-set conditions thereby indicating a malfunction of the washing unit.

Preferably, the one or more control switches are each selected from: manually operable control switches; remotely operable control switches.

Desirably, the concurrent compliance with the pre-set conditions is such that a condition applied to the received measurement signals is conditional upon the received monitoring signals.

The method may include sensing the water flow rate issuing the warning signal if the measurement signal indicates a non-zero water flow rate and concurrently a said monitoring signal(s) indicates that no said water outlet is in fluid communication with the water inlet.

The method may include sensing the water flow rate and issuing the warning signal if the measurement signal indicates no water flow and concurrently a said monitoring signal(s) indicates that a said water outlet is in fluid communication with the water inlet.

The method may include providing a first said water outlet and a second said water outlet, and sensing the water flow rate and issuing the warning signal if the measurement signal indicates a water flow rate exceeding: a first pre-set water flow rate threshold value and concurrently a said monitoring signal(s) indicates that only one of the first and second water outlets is in fluid communication with the water inlet; or, a second pre-set water flow rate threshold value exceeding the first pre-set water flow rate threshold value and concurrently a said monitoring signal(s) indicates that both of the first and second water outlets is in fluid communication with the water inlet.

The first pre-set water flow rate threshold value is preferably between 1 liter per minute and 12 liters per minute, or between 6 liters per minute and 12 liters per minute. The second pre-set water flow rate threshold value is preferably be one of: between 60 liters per minute and 100 liters per minute; between 30 liters per minute and 60 liters per minute; at least 100 liters per minute.

The method may include sensing the water pressure and issuing the warning signal if the measurement signal indicates a water pressure exceeding a pre-set upper pressure threshold value and concurrently a said monitoring signal(s) indicates that no said water outlet is in fluid communication with the water inlet.

The method may include sensing the water pressure and issuing the warning signal if the measurement signal indicates a water pressure less than a pre-set lower pressure threshold value, and concurrently a said monitoring signal(s) indicates that no said water outlet is in fluid communication with the water inlet.

The invention may provide an apparatus and method for remote user ID capture and remote transmission to First Responders.

In a fourth aspect, the invention may provide an emergency washing apparatus adapted for generating a controlled spray or jet of water directed at a user for washing therewith, the apparatus comprising: a washing unit including a water inlet and one or more water outlets arranged for forming the spray or jet of water received from the water inlet; a user identity apparatus configured to generate identification information identifying a user of the washing unit and to transmit the user identification information; a shower monitoring unit remote from the washing unit responsive to receipt of the transmitted user identification information to issue a telecommunications signal conveying: information identifying the location of the washing unit; and, information identifying the user.

Desirably, the user identity apparatus comprises any one or more of: a fingerprint detector configured for detecting a fingerprint of a user of the washing unit; a camera configured for detecting the face of a user of the washing unit; an RFID detector configured for detecting an RFID tag worn by a user of the washing unit. Facial recognition software may be used with the camera configured for detecting the face of a user of the washing unit, by applying the software to an image of the user as captured by the camera. Software may be used such as is readily apparent and available to the person of ordinary skill in the art.

Preferably, the washing unit includes a manually operable switch mechanism configured for activating the formation of the spray or jet of water, and the user identity apparatus includes the fingerprint detector arranged upon the manually operable switch and configured detect a fingerprint of a user in contact therewith.

Desirably, the shower monitoring unit includes a data processor and a display unit configured to generate one or each of: geographical coordinates identifying the location of the washing unit; a map of an environment local to the washing unit upon which the location of the washing unit is shown, and to display the geographical coordinates or map on the graphical user interface. For example, the geographical coordinates may then be used to show the location of the washing unit on an online mapping application/software (e.g. Google Maps).

Preferably, the shower monitoring unit is configured to generate one or each of: geographical coordinates identifying the location of the washing unit; a map of an environment local to the washing unit upon which the location of the washing unit is shown, and to convey the geographical coordinates or the map within the information identifying the location of the washing unit in the telecommunications signal. For example, the geographical coordinates may then be used to show the location of the washing unit on an online mapping application/software (e.g. Google Maps).

The shower monitoring unit may be configured to generate information selected from any one or more of: directions to the washing unit; medical data associated with the identity of the user, and to convey the information within the telecommunications signal.

In a corresponding aspect, the invention may provide an emergency washing method adapted for generating a controlled spray or jet of water directed at a user for washing therewith, the method comprising: providing a washing unit including a water inlet and one or more water outlets arranged for forming the spray or jet of water received from the water inlet; providing a user identity apparatus and therewith generating identification information identifying a user of the washing unit and transmitting the user identification information; providing a shower monitoring unit remote from the washing unit, and therewith receiving the transmitted user identification information and issuing a telecommunications signal conveying: information identifying the location of the washing unit; and, information identifying the user.

Desirably, the user identity apparatus comprises any one or more of: a fingerprint detector configured for detecting a fingerprint of a user of the washing unit; a camera configured for detecting the face of a user of the washing unit; an RFID detector configured for detecting an RFID tag worn by a user of the washing unit.

Preferably, the method includes providing a manually operable switch mechanism configured for activating the formation of the spray or jet of water, and providing a fingerprint detector upon the manually operable switch, and detecting a fingerprint of a user in contact therewith.

Desirably, the method includes providing a data processor and therewith generating one or each of: geographical coordinates identifying the location of the washing unit; a map of an environment local to the washing unit upon which the location of the washing unit is shown, and displaying the geographical coordinates or map on the graphical user interface. For example, the geographical coordinates may then be used to show the location of the washing unit on an online mapping application/software (e.g. Google Maps).

Preferably, the method includes generating one or each of: geographical coordinates identifying the location of the washing unit; a map of an environment local to the washing unit upon which the location of the washing unit is shown, and conveying the geographical coordinates or map within the information identifying the location of the washing unit in the telecommunications signal. For example, the geographical coordinates may then be used to show the location of the washing unit on an online mapping application/software (e.g. Google Maps).

The method may include generating information selected from any one or more of: directions to the washing unit; medical data associated with the identity of the user, and to convey the information within the telecommunications signal.

The invention may provide an apparatus and method for remote transmission of a geolocation of an emergency washing unit to First Responders.

In a fifth aspect, the invention may provide an emergency washing system adapted for generating a controlled spray or jet of water directed at a user for washing therewith, the apparatus comprising: a washing unit including a water inlet and one or more water outlets arranged for forming the spray or jet of water received from the water inlet; a positioning unit configured to generate positioning information identifying a location of the washing unit and to transmit the positioning information; a shower monitoring unit remote from the washing unit responsive to receipt of the transmitted positioning information to issue a telecommunications signal conveying information identifying the location of the washing unit.

The emergency washing apparatus may include a geo-spatial positioning transceiver unit operably connected to a Global Navigation Satellite System (GNSS) or a terrestrial wireless telecommunications network, and configured to determine the position of the washing unit accordingly.

The emergency washing apparatus may include user identity apparatus configured to generate identification information identifying a user of the washing unit and to transmit the user identification information; wherein the shower monitoring unit is responsive to receipt of the transmitted user identification information to issue a telecommunications signal conveying: information identifying the location of the washing unit; and, information identifying the user.

Desirably, the user identity apparatus comprises any one or more of: a fingerprint detector configured for detecting a fingerprint of a user of the washing unit; a camera configured for detecting the face of a user of the washing unit; an RFID detector configured for detecting an RFID tag worn by a user of the washing unit.

The washing unit may include a manually operable switch mechanism configured for activating the formation of the spray or jet of water, and the user identity apparatus may include the fingerprint detector arranged upon the manually operable switch and configured detect a fingerprint of a user in contact therewith.

Desirably, the shower monitoring unit includes a data processor and a display unit configured to generate one or both of: geographical coordinates, e.g. latitude and longitude, identifying the location of the washing unit; a map of an environment local to the washing unit upon which the location of the washing unit is shown, and to display the map, and/or the geographical coordinates, on the graphical user interface. For example, the geographical coordinates may then be used to show the location of the washing unit on an online mapping application/software (e.g. Google Maps).

Preferably, the shower monitoring unit is configured to generate one or both of: geographical coordinates, e.g. latitude and longitude, identifying the location of the washing unit; a map of an environment local to the washing unit upon which the location of the washing unit is shown, and to convey the geographical coordinates, and/or the map, within the information identifying the location of the washing unit in the telecommunications signal. For example, the geographical coordinates may then be used to show the location of the washing unit on an online mapping application/software (e.g. Google Maps).

The shower monitoring unit may be configured to generate information selected from any one or more of: directions to the washing unit; medical data associated with the identity of the user, and to convey the information within the telecommunications signal.

In a corresponding aspect, the invention may provide an emergency washing method adapted for generating a controlled spray or jet of water directed at a user for washing therewith, the method comprising: providing a washing unit including a water inlet and one or more water outlets arranged for forming the spray or jet of water received from the water inlet; providing a positioning unit and therewith generating positioning information identifying a location of the washing unit and transmitting the positioning information; providing a shower monitoring unit remote from the washing unit and therewith receiving the transmitted positioning information and issuing a telecommunications signal conveying information identifying the location of the washing unit.

The method may include providing a geo-spatial positioning transceiver unit operably connected to a Global Navigation Satellite System (GNSS) or a terrestrial wireless telecommunications network, and therewith determining the position of the washing unit accordingly. Examples of a Global Navigation Satellite System (GNSS) include the following: The United States' Global Positioning System (GPS); the Russian Federation's Global Orbiting Navigation Satellite System (GLONASS).

The method may include providing user identity apparatus and therewith generating identification information identifying a user of the washing unit, and transmitting the user identification information; and by the shower monitoring unit, responding to receipt of the transmitted user identification information by issuing a telecommunications signal conveying: information identifying the location of the washing unit; and, information identifying the user.

Desirably, the user identity apparatus comprises any one or more of: a fingerprint detector configured for detecting a fingerprint of a user of the washing unit; a camera configured for detecting the face of a user of the washing unit; an RFID detector configured for detecting an RFID tag worn by a user of the washing unit.

Preferably, the method includes providing a manually operable switch mechanism configured for activating the formation of the spray or jet of water, and providing a fingerprint detector upon the manually operable switch, and detecting a fingerprint of a user in contact therewith.

Desirably, the method includes providing a data processor and therewith generating one or both of: geographical coordinates, e.g. latitude and longitude, identifying the location of the washing unit; a map of an environment local to the washing unit upon which the location of the washing unit is shown, and displaying the map and/or the geographical coordinates on the graphical user interface. For example, the geographical coordinates may then be used to show the location of the washing unit on an online mapping application/software (e.g. Google Maps).

Preferably, the method includes generating one or both of: geographical coordinates, e.g. latitude and longitude, identifying the location of the washing unit; a map of an environment local to the washing unit upon which the location of the washing unit is shown, and conveying the map and/or the geographical coordinates within the information identifying the location of the washing unit in the telecommunications signal. For example, the geographical coordinates may then be used to show the location of the washing unit on an online mapping application/software (e.g. Google Maps).

The method may include generating information selected from any one or more of: directions to the washing unit; medical data associated with the identity of the user, and to convey the information within the telecommunications signal.

In another aspect, the invention provides an emergency washing system comprising a washing unit including a water tank for containing water, a water outlet comprising a shower head and/or a nozzle for outputting a spray or jet of water from the water tank, a water flow control part operable and arranged to reversibly place the water tank in fluid communication with the water outlet in response to either of: manual control of the washing unit, or a remote control signal. The washing unit also includes a sensor part configured to transmit monitoring signals each indicating a respective occurrence of said fluid communication. The emergency washing system further comprises a shower control unit which is remote from the washing unit and which is arranged to receive the monitoring signals and to issue the remote control signal to the water flow control part if an interval of time since receipt of the most recently received monitoring signal exceeds a threshold time interval value.

The emergency washing system preferably also includes a flow-rate sensor arranged to measure a rate of flow of water from the water tank to the water outlet. The monitoring signal preferably includes a measured rate of flow associated with a respective occurrence of the fluid communication. The shower control unit is preferably arranged to issue a warning signal if the measured rate of flow falls below a threshold water flow rate value.

The emergency washing system may include a wearable user identity tag containing user identification information configured for detection. The washing unit may comprise a detector responsive to proximity of the user identity tag to: detect the user identification information contained therein and, transmit the detected user identification information to the shower control unit. Desirably, the shower control unit is responsive to receipt of the transmitted user identification information to issue a telecommunications signal conveying information identifying the location of the washing unit, and information identifying the user.

In yet another aspect, the invention provides an emergency washing method in an emergency washing system comprising a washing unit including: a water tank for containing water, a water outlet comprising a shower head and/or a nozzle for outputting a spray or jet of water from the water tank, a water flow control part, and a sensor part; and a shower control unit remote from the washing unit. The method comprises the following steps performed by the components of the emergency washing system, as follows. By the water flow control part, reversibly placing the water tank in fluid communication with the water outlet in response to either of: manual control of the washing unit, or a remote control signal. By the sensor part, transmitting monitoring signals each indicating the sensing thereby of a respective occurrence of said fluid communication. By the shower control unit, receiving the monitoring signals and issuing the remote control signal to the water flow control part if an interval of time since receipt of the most recently received monitoring signal exceeds a threshold time interval value.

Desirably, in the emergency washing method the emergency washing system comprises a flow-rate sensor, and the method includes the following steps. By a flow-rate sensor, measuring a rate of flow of water from the water tank to the water outlet, wherein a said monitoring signal includes a said measured rate of flow associated with a respective occurrence of said fluid communication. By the shower control unit, issuing a warning signal if the measured rate of flow falls below a threshold water flow rate value.

The method may include providing a wearable user identity tag containing user identification information configured for detection and, at the washing unit, providing a detector responsive to proximity of the user identity tag and therewith detecting the user identification information contained therein. The method preferably includes transmitting the detected user identification information to the shower control unit and, by the shower control unit, in response to receipt of the transmitted user identification information, issuing a telecommunications signal conveying information identifying the location of the washing unit and information identifying the user.

It is to be understood that the invention according to any one aspect described above may also comprise the features of the invention described according to any one or more of the other aspects described above.

The user identity apparatus, described above in any aspect, may comprise any one or more of: a fingerprint detector configured for detecting a fingerprint of a user of the washing unit; a camera configured for detecting the face of a user of the washing unit; an RFID detector configured for detecting an RFID tag worn by a user of the washing unit. Facial recognition software may be used with the camera configured for detecting the face of a user of the washing unit, by applying the software to an image of the user as captured by the camera. Software may be used such as is readily apparent and available to the person of ordinary skill in the art.

### BRIEF DESCRIPTION OF DRAWINGS

For a better understanding of the invention, and without intending to limit the scope of the invention, there now follows a description of some examples and embodiments of the invention with reference to the drawings, of which:

FIG. 1 shows a schematic diagram illustrating an emergency washing system according to an embodiment of the invention;

FIG. 2 shows a schematic diagram illustrating an emergency washing system according to an embodiment of the invention;

FIG. 3 shows a user interface display of water flowrate, pressure and temperature data collected over an interval of time by the emergency washing system according to FIG. 1 or FIG. 2;

FIG. 4A shows a user interface display of the status and spatial location of a plurality of the emergency washing systems each of which is according to FIG. 1 or FIG. 2;

FIG. 4B shows a user interface display of the status and spatial location of a plurality of the emergency washing systems each of which is according to FIG. 1 or FIG. 2;

FIG. 5 shows a flow chart of steps in an emergency washing method in an emergency washing system according to FIG. 1 or FIG. 2;

FIG. 6A shows a further flow chart of steps in an emergency washing method in an emergency washing system according to FIG. 1 or FIG. 2;

FIG. 6B shows another flow chart of steps in an emergency washing method in an emergency washing system according to FIG. 1 or FIG. 2;

FIG. 7 shows a flow chart of steps in an emergency washing method in an emergency washing system according to FIG. 1 or FIG. 2;

### DESCRIPTION OF EMBODIMENTS

In the drawings, like items are assigned like reference symbols.

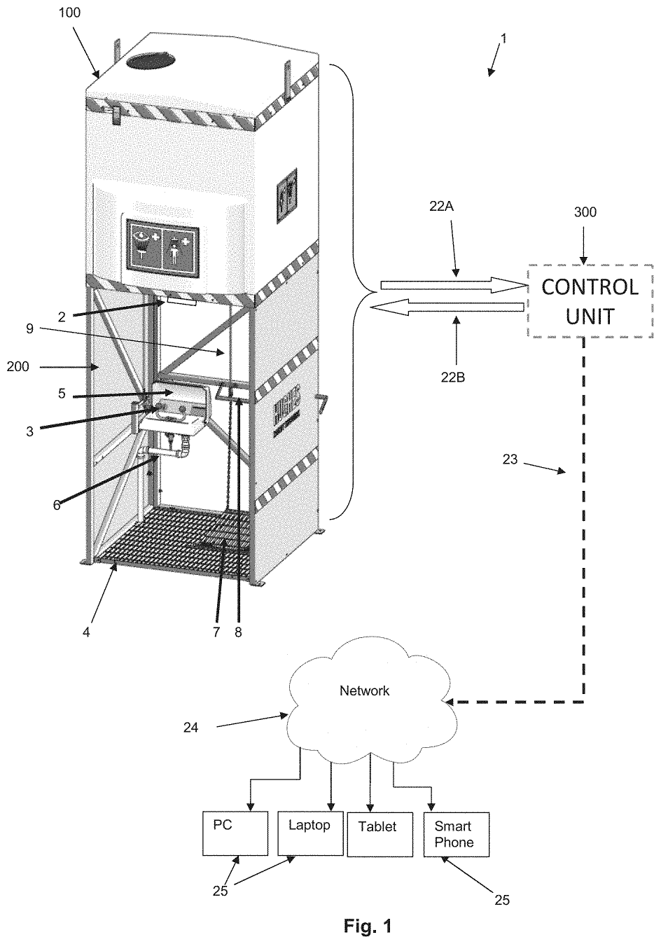

Referring to FIG. 1, there is illustrated, in schematic form, an emergency washing system according to an embodiment of the invention. The emergency washing system includes a washing unit which comprises a water tank (100) and a washing cubicle (200) above which the water tank is disposed such that the floor of the water tank provides the ceiling of the washing cubicle. The water tank is configured to contain clean water for use in washing a user of the shower within the washing cubicle and, for this purpose, two separate water gravity-fed water outlets are disposed below the water tank within the washing cubicle, each of which is in fluid communication with the water tank.

A first of these two water outlets comprise a shower head (2) disposed in the ceiling of the shower cubicle which is configured to produce a downward shower spray pattern of water fed to it from the water tank, in use, directed towards the floor (4) of the washing cubicle. A second of the two water outlets is a nozzle unit (3) for outputting a pair of simultaneous upward sprays or jets of water from the water tank, at an eye-washing station located within the washing cubicle at about waist height (e.g. between 1.0 m and 1.25 m above ground level) and is connected in fluid communication with the water tank (100) via a water pipe line (6) which passes from the water tank to the nozzle unit (3). The nozzle unit is configured to produce two sprays/jets laterally separated by a typical human inter-ocular distance, and of substantially the same water output flow rate and pressure such that the water sprays/jets are of substantially the same shape, form and force. This permits both eyes of a user to be bathed in water simultaneously at the same rate of water flow to each eye.

The washing unit also includes water outlet control parts comprising a water outlet control valves (not shown) controllable to open and close to reversibly place the water tank in fluid communication with the shower head (2) and/or the eye-washing nozzle (3). The water outlet control parts are arranged to operate in this way in response to either of: a local, manual control operation of shower/nozzle on/off controls of the washing unit situated within the washing cubicle to manually switch on/off a flow of water; or a remote control signal issued remotely from the washing unit to the same effect.

In particular, the washing unit comprises a number of manual control parts (5, 7, 8, 9) contained within the washing cubicle and configured for manual operation by a user there. Each of the manual control parts is manually operable, in the manner of an on-off switch, to operate either a shower water valve switch (not shown) or an eye/face wash water valve switch (not shown). For example, the shower valve switch is mechanically connected to each of: a foot peddle (7); a hand lever (8); a pull-chain (9); such that manual operation of any of these devices will operate the shower water valve switch to reversibly place the shower head (2) in fluid communication with the water tank, or to terminate such fluid communication, depending upon whether or not the two are already in fluid communication when the switch is operated. Similarly, the eye/face wash valve is mechanically connected to a cover panel (5) manually pivotable to reveal the eye-washing nozzles such that manual operation of the cover panel will operate the eye/face wash water valve switch to reversibly place the eye-washing nozzle (3) in fluid communication with the water tank, or to terminate such fluid communication, depending upon whether or not the two are already in fluid communication when the switch is operated.

The washing unit also includes a plurality of sensor parts each configured to transmit monitoring signals which indicate a respective occurrence of the operation of an associated water valve switch for causing fluid flow from the water tank (100) to a respective one of the shower head (2) or the eye-wash nozzle unit (3). Each sensor is configured to detect when a valve switch is operated on/off via manual operation of a respective one of the manual control parts (5, 7, 8, 9). A sensor may be any suitable sensor for detecting the operation of a switch, such as may be readily available to the person skilled in the art. For example, each switch may comprise mechanical movement of a lever to open/close an associated valve, and the associated sensor may comprise a permanent magnet fixed to the lever so as to me moveable into/away-from proximity to a static magnet sensor responsive to detect the permanent magnet when in said proximity. Accordingly, detection or non-detection of the presence of the permanent magnet is indicative of whether the lever arm has been actuated to open or close the associated valve.

The washing unit also includes a flow-rate sensor assembly (not shown) arranged to measure a rate of flow of water from the water tank to the shower head (2) and to the eye-wash nozzle unit (3).

The washing unit may comprise any one or more of the following sensors: (a) An ambient temperature sensor for measuring the ambient temperature around the washing unit; (b) A water level sensor for sensing the level of water in the water tank (100); (c) A water tank temperature sensor for measuring the temperature of water in the water tank; (d) An ambient light-level sensor for measuring the ambient light level at the washing unit; (e) A water inlet flow rate detector for measuring a rate of flow of water (e.g. refill water supply) into the water tank (100); (f) An inlet temperature sensor for measuring the temperature of water at the water inlet of the water tank; (g) A switch sensor for sensing operation of (or the status: ‘on’/′off) of a valve control switch coupled to the shower water valve; (h) A switch sensor for sensing operation of (or the status: ‘on’/‘off’) of a valve control switch coupled to the eye-wash water valve; (i) A flow-rate sensor arranged to measure a rate of flow of water from the water tank to the shower head (2) and/or to the eye-wash nozzle unit (3); and (j) A water pressure sensor arranged to measure the water pressure at the water outlet of the water tank.

The emergency washing system further comprises a shower control unit (300) which is located at a geographically remote location separated from the geographical location of the washing unit (100, 200). The shower control unit is arranged to receive the monitoring signals (22A) issued by the sensor part located within the washing unit (100, 200) and to generate and issue the remote control signal (22B) to the water flow control parts as appropriate.

For example, if more than a predetermined time has passed between uses of the washing apparatus (100, 200), then as a safety operation, the appropriate water flow control valves of the washing unit are remotely controlled to flush water from the water tank and through the shower head (2) and to the eye-wash nozzle unit (3). This not only serves to prevent stagnation of water in the system and the dangers that can bring, as discussed above, but also to allow measurement and/or monitoring signals to be remotely collected by the control unit (300) from any of the sensors arrayed within the washing unit (100).

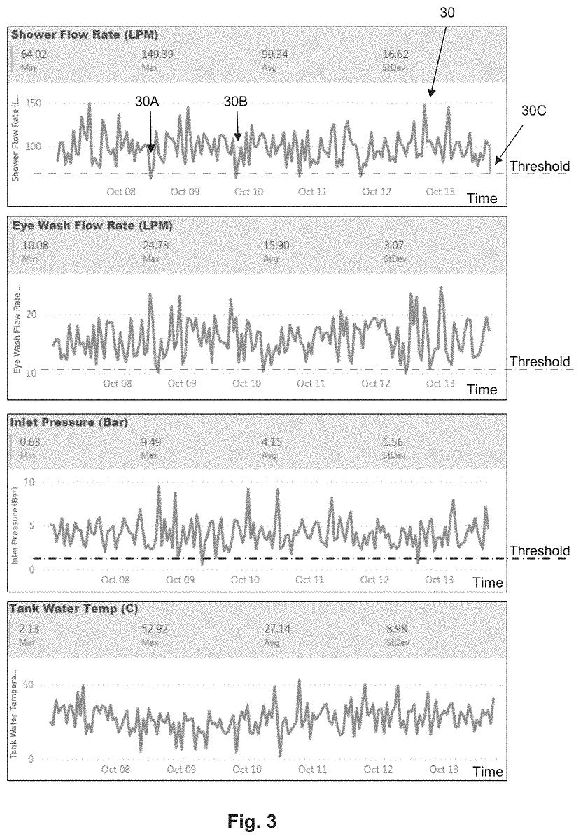

The measurement/monitoring signals (22A) include, for example, a measured rate of flow of water associated with the occurrence of fluid communication between the water tank (100) and one or both of the water outlets (2, 3). FIG. 3 schematically shows an example of a time-sequence (30) of such measurement values and may represent the flow rate of water when e.g. the shower valve switch is open/on but the eye/face wash valve switch is closed/off, or vice versa, or e.g. when both the shower valve switch is open/on but the eye/face wash valve switch is open/on. This means that the measured flow rate may correspond to the rate of flow of water to the user e.g. via the shower head (2) alone, but not via the eye-wash nozzle unit (3), or vice versa. Of course, when the shower main valve switch is closed/off but the eye/face wash valve switch is open/on, then the measured flow rate may correspond to the rate of flow of water to the user via the eye-wash nozzle unit (3). The detected water flow rate is the flow rate detected over a continuous time interval (e.g. 1.5 minutes).

The shower control unit (300) is arranged to issue a warning signal (23) if the measured rate of flow falls below a threshold water flow rate value. As can be seen from FIG. 3, over the time period in question, the measured flow rate, measured while the shower main valve switch is open/on, varies over the time interval and falls below the threshold value twice. The first occurrence (30A) is shortly after the beginning of the interval of time, just after the shower is switched on, and the second occurrence (30B) is approximately mid-way through the time interval. A third occurrence (30C) of below-threshold water flow rate occurs when the time interval ends as the shower is switched off by the user and the shower head (2) is no longer in fluid communication with the water tank (100). Of these three occurrences of below-threshold water flow rate, the first two occurrences (30A, 30B), cause the control unit to issue a warning signal (23). However, because the third occurrence of a below-threshold water flow rate does not happen when the shower head (2) is in fluid communication with the water tank (100), then the condition for issuing a warning signal is not met and no warning signal is issued. Thus, a condition required for issuance of a warning signal are that a below-threshold water flow rate measurement is received and that this flow rate measurement is concurrent with the water tank and water outlet (shower head or nozzle) being in fluid communication with each other.

The warning signals (23) are transmitted as telecommunications signals via a communications network (24), e.g. the Internet, to a systems operator, manager or other responsible personnel in charge of maintaining the emergency washing system. In particular, the warning signal is received at the communications device (25) used by the personnel in question. The communications device may be a desk-top computer, Tablet or Smartphone.

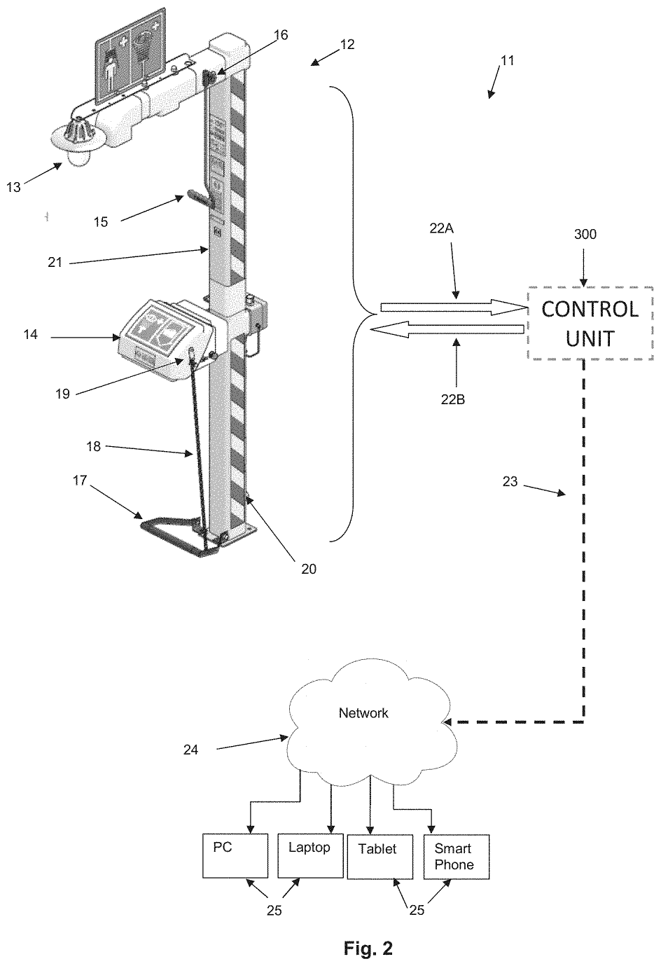

Referring to FIG. 2, there is shown an emergency shower apparatus (11) which has the same function and features as the emergency shower apparatus described with reference to FIG. 1, except for the provision of a mains water supply inlet at the base of the washing unit (12) in place of a water tank placed above the washing cubicle of the washing unit of FIG. 1. In addition, the washing unit of FIG. 2 comprises a stand-alone shower and eye-wash assembly with no washing cubicle. In particular, the washing unit (12) comprises a mains water inlet (20) located at the base of a column (21) housing water conduits leading from the mains water inlet to an eye-washing nozzle assembly housed underneath a flip-top cover (14), and to a shower head (13). The eye-washing nozzle assembly is served by a water flow control valve (not shown) housed within the washing unit in fluid communication with the mains water inlet (20) and is manually operable via a manual control switch mechanism (17, 18, 19) to open/close to place the eye-washing nozzle assembly in fluid communication with the mains water inlet (20). The manual control switch mechanism comprises a foot peddle (17) connected to a valve switch (19) via a pull-chain. Depression of the foot peddle by a user causes that pull-chain to pull down on the valve switch to operate it (e.g. open the valve). Similarly, the shower head (13) is served by a water flow control valve (not shown) housed within the washing unit in fluid communication with the mains water inlet (20) and is manually operable via a manual control switch mechanism (15) to open/close to place the shower head in fluid communication with the mains water inlet (20). The manual control switch mechanism comprises a pull-handle (15) connected to a valve switch (16) via a pull-rod. Downward pulling of the pull handle by a user causes that pull-rod to pull down on the valve switch (16) to operate it (e.g. open the valve).

All other functions, functionality and interactions (22A, 22B) of this washing unit (12) with the control unit (300) are as described herein with reference to FIG. 1. In particular, the interaction (23) and functionality of the control unit (300) shown in FIG. 2, with networks (24) and communications devices (25) are as described herein with reference to FIG. 1.

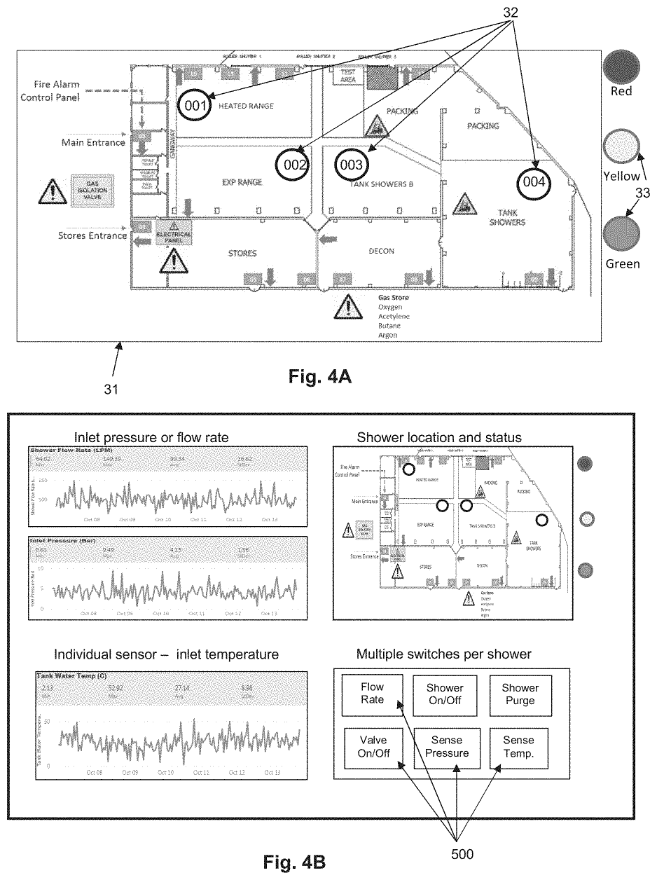

The communications device includes a display screen upon which the measurement data (30) may be displayed graphically as shown in FIG. 4A. Furthermore, the communications device may be configured to display a schematic map of the geographical area within which the washing unit (100, 200) is situated, and in which the location and status of the washing unit is visually indicated. An example of this is illustrated in FIG. 4A in which the geographical area is the floor of a chemical processing plant and the schematic map of that illustrates a map of the various chemical processing assemblies and units of the plant together with the separate locations of nine separate emergency washing units (unit numbers: 001, 002, . . . 009) each of which is according to the invention e.g. such as is shown in FIG. 1 or FIG. 2.

A respective colored circle (32) surrounds the immediate location, upon the map, of a washing unit, together with the associated unit number. The color (33) of a given circle indicates whether a new warning signal has been received from the washing unit associated with that map location and unit number, and either has or has not yet been investigated/corrected. In the example given in FIG. 4A, a red color indicates that a new warning signal has been received and remains un-investigated/corrected, while a green color indicates that no new warning signal has been received. A yellow color indicates that a new warning signal has been received and is currently under investigation for maintenance/correction.

The emergency washing system may also include a wearable user identity tag (not shown) containing user identification information configured for detection. An example would be an RFID tag worn by an employee or personnel present on the plant floor and tasked with operating the plant machinery. The washing unit comprises an RFID detector unit (not shown) located within the washing cubicle (200; FIG. 1), or the free-standing shower column (12, FIG. 2) and is configured to be responsive to proximity of the user identity tag when the user is within the washing cubicle, or shower column. The RFID detector unit is configured to detect the user identification information contained within the RFID tag, and to transmit (22A) the detected user identification information to the shower control unit (300). The shower control unit (300) is responsive to receipt of the transmitted user identification information to issue a telecommunications signal (23) conveying information identifying the washing unit in question (e.g. see FIG. 4A, FIG. 4B: unit #001, or #004 etc.) and the location of the washing unit (e.g. directions/coordinates according to the map of FIG. 3), and information identifying the user.

The telecommunications signal is transmitted via a telecommunications network (24), such as the internet, to the communications device(s) of First-Responder (e.g. first aid) personnel associated with the chemical plant in which the washing unit (and the user of it) is located, and also (or alternatively) to the communications device(s) of ambulance/emergency-services personnel. The information identifying the user may include relevant (possibly life-critical) medical information particular to the identified user, such as the equipment operated by the user within the plant (e.g. the particular dangerous chemicals he/she may have been exposed to), any medical allergies he/she may have, any existing medical conditions, his/her gender, age and other medical information which may assist first aid and/or ambulance personnel in more effectively helping the identified user of the emergency shower unit.

FIG. 4B illustrates a graphical user interface (e.g. control ‘dashboard’) of a communications device of a systems operator, manager or other responsible personnel in charge of maintaining the emergency washing system. In particular, the monitoring/measurement signals (22A) are received at the communications device (25) used by the personnel in question. The communications device may be a desk-top computer, an iPad or an iPhone. The communications device comprises processor unit configured to receive the data conveyed by the monitoring/measurement signals and to generate a graphical user interface (GUI) configured to display the data graphically as shown in FIG. 4B. For example, the received data may comprise time-series data relating to water pressure and/or water flow rate measurements, and water temperature. The GUI may also comprise one or more control switches presented as buttons (500) each responsive to a cursor selection/‘click’ upon the GUI to cause the control unit (300) to issue a specific control signal (22B) to the washing unit, such as to control the water flow control valves thereof.

In general, the embodiments of the invention illustrated in FIG. 1 and FIG. 2 provides an emergency washing apparatus adapted for generating a controlled spray or jet of water directed at a user for washing with. The apparatus has a washing unit which may be as shown in FIG. 1 or as shown in FIG. 2, or another arrangement, each including at least a water inlet for receiving water (e.g. from a tank as in FIG. 1, or from a mains water supply as in FIG. 2) and one or more water outlets (e.g. a shower head and an eye-wash nozzle apparatus) arranged for forming the spray or jet of water received from the water inlet.

In preferred embodiments, such as those described above with reference to FIG. 1 or FIG. 2, the apparatus includes one or more sensors configured to sense one or more properties of the water received at the water inlet. These properties may be any one or more selected from: a water flow rate (using a flow rate sensor); a water pressure (using a pressure sensor); a water temperature (using a temperature sensor); a water pH level (using a pH sensor), a bacteria content (using a bacteria detector). The detectors used for this purpose may be any suitable detector such as would be readily apparent and available to the person of ordinary skill in the art.

In preferred embodiments, such as those described above with reference to FIG. 1 or FIG. 2, the one or more sensors are configured to generate a respective measurement signal indicating a value of the one or more sensed properties of the water, and to transmit the measurement signal for reception remotely from the washing unit. The transmitted measurement signal may be transmitted electronically, optically (e.g. fiber-optically) or wirelessly (e.g. a radio/telecoms signal). The transmission may be received by the control unit described above for use in monitoring and/or implementing control operations upon the washing unit. For example, the shower control unit may be arranged to issue a warning signal if the received measurement signals fail to comply with respective pre-set conditions thereby indicating a malfunction of the washing unit. The shower control unit may be arranged to generate a graphical user interface (GUI) arranged to display the measurements graphically on a display unit (e.g. a PC, or laptop etc.).

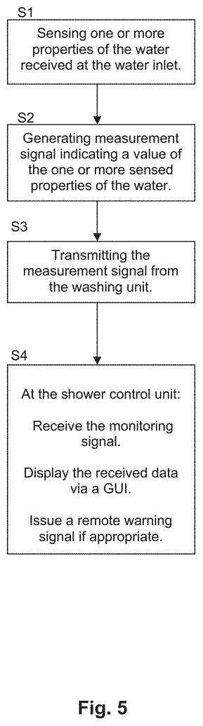

For example, FIG. 5 illustrates the steps in an emergency washing method implemented by the emergency washing system described above. The method comprises the following steps performed by the components of the emergency washing system, as follows.

In a first step S1: Sensing one or more properties of the water received at the water inlet.

In a second step S2: Generating measurement signal indicating a value of the one or more sensed properties of the water.

In a third step S2: Transmitting the measurement signal from the washing unit.

In a fourth step S4: By the shower control unit: receiving the monitoring signal and/or displaying the received data via a GUI and/or issuing a remote warning signal if appropriate.

In preferred embodiments, one or more remotely controllable water flow control valves of the washing unit (FIG. 1 or FIG. 2) are each remotely controlled to reversibly place a respective the water outlet (e.g. a shower head, an eye-wash nozzle) in fluid communication with the water inlet (e.g. water tank outlet, mains water supply input port). In these embodiments, the shower control unit may be arranged to remotely detect when the water flow control valves are locally, manually operated (opened) by a user of the washing unit and therefore remote operation of the valves is not necessary. Alternatively, the shower control unit may be arranged to remotely control the one or more water flow control valves to reversibly place a respective water outlet in fluid communication with the water inlet. In some embodiments, a water flow-rate sensor is arranged to measure a rate of flow of water from the water inlet to the one or more water outlets, and to generate a monitoring signal indicating a measured rate of flow. The shower control unit is then arranged to perform remote data gathering by controlling the remotely controllable water flow control valves to place a respective water outlet in fluid communication with the water inlet for a finite pre-set time period. The shower control unit is arranged to receive the monitoring signals during the time period.

The shower control unit may issue a warning signal if the measured rate of flow falls below a pre-set threshold water flow rate value, and may repeat the remote data gathering if an interval of time since the most recent previous said remote data gathering exceeds a threshold time interval value.

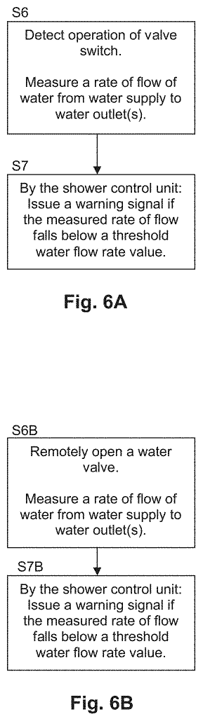

FIG. 6A illustrates the steps in an emergency washing method implemented by the emergency washing system described above. The method comprises the following steps performed by the components of the emergency washing system, as follows.

In a first step S6: By the emergency shower unit, detect operation of a water flow control valve switch, and in response, measure a rate of flow of water from the water supply (e.g. water tank) to the water outlet (shower head or eye-wash nozzle).

In a second step S7: By the shower control unit, issue a warning signal if the measured rate of flow falls below a threshold water flow rate value.

This methodology applies when the water flow control valves are locally, manually operated (opened) by a user of the washing unit and therefore remote operation of the valves is not necessary.

FIG. 6B illustrates the steps in an emergency washing method implemented by the emergency washing system described above. The method comprises the following steps performed by the components of the emergency washing system, as follows.

In a first step S6B: By the emergency shower unit, remotely operate a water flow control valve switch to ‘open’ it, and subsequently, measure a rate of flow of water from the water supply (e.g. water tank) to the water outlet (shower head or eye-wash nozzle).

In a second step S7B: By the shower control unit, issue a warning signal if the measured rate of flow falls below a threshold water flow rate value.

This methodology applies when the water flow control valves are not locally, manually operated (opened) by a user of the washing unit and therefore remote operation of the valves is necessary.

The methodology, according to FIG. 6A or FIG. 6B, may include sensing one or more properties of the water such as a water flow rate, and/or a water pressure, and the shower control unit issue a warning signal if the received measurement signals fail to comply with pre-set conditions thereby indicating a malfunction of the washing unit. For example, the pre-set conditions may require that if all water flow control valves are ‘closed’, then the water flow measurement indicates no water flow. For example, the pre-set conditions may require that if one, but not all, water flow control valves are ‘open’, then the measured flow rate should be above a pre-set flow rate and/or a measured water pressure is within a pre-set pressure range.

The pre-set rates/ranges may be specific to which of the water flow control valves are open and which are closed. This mirrors that requirement that required water flow rates and pressures differ depending on which one of the shower and the eye-wash apparatus is in use, or if both are in use.

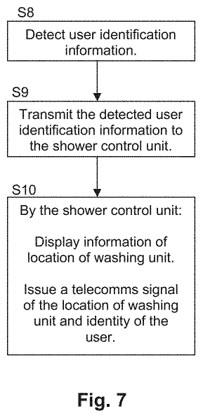

FIG. 7 illustrates the steps in an emergency washing method implemented by the emergency washing system described above. The method comprises the following steps performed by the components of the emergency washing system, as follows.

In a first step S8: By the emergency shower unit, detect user identification information.

In a second step S9: By the emergency shower unit, transmit the detected user identification information to the shower control unit.

In a third step S10: By the shower control unit, issuing a telecommunications signal conveying the location of the washing unit and identity of the user.

It will be appreciated that the examples of embodiments of the invention, described above, include examples of decision logic applied by the control unit to remote measurements taken from the washing unit (FIG. 1, FIG. 2). These examples are not intended to be exhaustive, and other decision logic operations may be applied by the control unit. Table 1, shown below, illustrates a more detailed set of possible measurements gathered by the control unit, according to preferred embodiments, and examples of the applied decision logic that may be applied to the measurements by the control unit, in the form: ‘If condition X occurs, then implement response Y’. Abbreviation ‘PIR’ refer to ‘Passive Infra-Red’.

TABLE 1

| | | Applied Decision | | Data |
|---|---|---|---|---|
| SENSOR | Purpose | Logic | Benefit | Captured |
| Water | Confirms tank | If water temp too | Audit log | Temperature/ |
| Temperature | water | low -trigger | confirming | time/date |
| | temperature is | heater/user | compliance | |
| | within | intervention | | |
| | EN/ANSI required | | | |
| | levels | | | |
| PIR-activated | Capture the person | If PIR sensor | Info can then be | On Demand/ |
| Camera | as they enter the | activated and | emailed to first | Jpeg Image/ |
| | shower | shower then | responders, | Time/Date |
| | | shower then send | audit log | |
| | | image to | created | |
| | | cloud | | |
| Water | Checking water PH | If PH levels out | If no action | PH Level/Water |
| quality | levels/bacteria | of range then | after set time | Quality/Time/ |
| | sensor | dump water/ | limit -water can | Date |
| | | prompt user | be dumped | |
| | | interaction | remotely from | |
| | | | tank | |
| Ambient | Temperature where | Data capture of | | Temperature/ |
| Temperature | the shower is | environment | | Time/Date |
| | located -general | | | |
| | monitoring | | | |
| Water level | Checking water | If water below | Always have | Water Level/ |
| | level in tank | threshold open | the right | Time/Date |
| | | water valve to fill - | amount of | |
| | | turn off when | water for the | |
| | | level achieved | 15 minute | |
| | | | rinsing | |
| | | | requirement | |
| Light sensor | Is the area around | if low light then | Data capture of | Lux/ |
| | the shower visible | trigger light to be | environment | Lumen Level |
| | ‘enough’ | turned on | | |
| Water Inlet | Check incoming | If water | Data capture/ | Temperature |
| Temperature | water supply level | temperature too | best practice | |
| Sensor | is within EN/ANSI | high then trigger | | |
| | levels | report | | |
| Shower | Sensing when | If shower operated | Trigger live | All Data |
| activation | shower is operated | then push data to | alert to site | Points |
| | | cloud | team | |
| Eye/face | Sensing when | If eyebath | Trigger live | On/Off |
| wash switch | eyebath is operated | operated then | alert to site | |
| | | push all data to | team | |
| | | cloud | | |
| Inlet pressure | Ensuring pressure | If pressure out of | Communicate | Pressure |
| sensor | is within | range then alert | potential issue | (BAR/PSI)/ |
| | the recommended | maintenance | to site | Time/Date |
| | working level for | | | |
| | the shower | | | |
| Flow switch | Sensing flow of | If water flow out | | Flow |
| | water to the | of preset minimum | | (Yes/No) |
| | shower or volume | (76 LPM) trigger | | |
| | of water moving | alert | | |
| | through the | | | |
| | system | | | |
| Shower user | Detect who is | If fingerprint | Get critical | User/ |
| profile | using the shower to | taken or RFID tag | information to | Health Record/ |
| | know more about | confirmed send | First | Allergies/ |
| | their medical | user data to first | Responders | Existing |
| | profile | responders | | Health Issues |
| | (confirm input) | | | |
| Geo | Confirm location of | If shower activated | Large sites | GPS |
| Location | tank shower | then location to be | would benefit | Coordinates |
| | | pushed to cloud | from this | |
| Shower valve | Remotely test/ | If weekly testing | Ensures | All Data |
| actuation | flush/activate the | not carried out | flushing of | Points |
| | shower | then prompt | system to | |
| | | alert/countdown | prevent | |
| | | timer to | bacteria/ | |
| | | automatically | legionella and | |
| | | flush shower - | compliance | |
| | | report back on | | |
| | | flow rate | | |
| Eyewash | Remotely test/ | If weekly testing | Ensures | All Data |
| valve | flush/activate | not carried out | flushing of | Points |
| actuation | the eyebath | then prompt | system to | |
| | | alert/countdown | prevent | |
| | | timer to | bacteria/ | |
| | | automatically | legionella and | |
| | | flush eyewash - | compliance | |
| | | report back on | | |
| | | flow rate | | |
| Alarm | Trigger remote | Alert multiple | | All Data |
| signal | alarms | people | | Points |
| | | instantaneously | | |
| Route to | Ability to direct | if activation then | Ensures speedy | Geo-Location |
| shower | people to | send route to First | response time | |
| | the user of the | Responders | | |
| | shower | | | |
| | (Smartphone | | | |
| | interface) | | | |

1. An emergency washing apparatus adapted for generating a controlled spray or jet of water directed at a user for washing therewith, the emergency washing apparatus comprising:
a washing unit including a water inlet and one or more water outlets arranged for forming said spray or jet of water received from the water inlet; and
one or more sensors configured to sense one or more properties of the water received at the water inlet selected from: a water flow rate, a water pressure, a water temperature, a water pH level, a bacteria content, and
wherein the one or more sensors are configured to generate a respective measurement signal indicating a value of the one or more sensed properties of the water, and to transmit the measurement signal for reception locally at, or remotely from, the washing unit.

2. The emergency washing apparatus of claim 1, including a shower control unit which is located locally at, or remote from, the washing unit and which is arranged to receive said respective measurement signals and to issue a warning signal if said received measurement signals fail to comply with respective pre-set conditions thereby indicating a malfunction of the washing unit.

3. The emergency washing apparatus of claim 2, including:
one or more remotely controllable water flow control valves each configured in fluid communication with the water inlet and with a respective one of said one or more water outlets, wherein each remotely controllable water flow control valve is operable to reversibly place a respective said water outlet in fluid communication with the water inlet;
wherein the shower control unit is arranged to remotely control said one or more remotely controllable water flow control valves to reversibly place a respective said water outlet in fluid communication with the water inlet.

4. The emergency washing apparatus of claim 3, in which the shower control unit is arranged to issue a said warning signal if a plurality of said received measurement signals fail to comply concurrently with pre-set conditions whereby concurrent compliance with said pre-set conditions is such that a condition applied to one said received measurement signals is conditional upon another said received monitoring signal.

5. The emergency washing apparatus of claim 4, wherein the shower control unit is located locally at, or remote from, the washing unit and which is arranged to receive said respective measurement signals, wherein the shower control unit includes a data processor and a display unit configured to generate and display a graphical user interface displaying one or more of said sensed properties of the water.

6. The emergency washing apparatus of claim 5, wherein the data processor and the display unit are configured to issue said warning signal as a warning displayed by said graphical user interface.

7. The emergency washing apparatus of claim 6, in which the washing unit includes a user identity unit configured to generate identification information identifying a user of the washing unit and to transmit the user identification information;
wherein the emergency washing apparatus includes a shower control unit located locally at, or remote from, the washing unit and responsive to receipt of the transmitted user identification information to issue a telecommunications signal conveying:
information identifying a location of the washing unit; and, information identifying the user.

8. The emergency washing apparatus of claim 7, wherein the data processor and the display unit are configured to generate one or each of: geographical coordinates identifying the location of the washing unit; a map of an environment local to the washing unit upon which the location of the washing unit is shown, and to display the geographical coordinates or the map on said graphical user interface.

9. An emergency washing apparatus adapted for generating a controlled spray or jet of water directed at a user for washing therewith, the emergency washing apparatus comprising:
a washing unit including a water inlet and one or more water outlets arranged for forming said spray or jet of water received from the water inlet;
one or more remotely controllable water flow control valves each configured in fluid communication with the water inlet and with a respective one of said one or more water outlets, wherein each remotely controllable water flow control valve is operable to reversibly place a respective said water outlet in fluid communication with the water inlet;
a shower control unit which is remote from the washing unit and which is arranged to remotely control said one or more remotely controllable water flow control valves to reversibly place a respective said water outlet in fluid communication with the water inlet.

10. The emergency washing apparatus of claim 9, including a water flow-rate sensor arranged to measure a rate of flow of water from said water inlet to said one or more water outlets, and to generate a monitoring signal indicating a measured rate of flow;
wherein the shower control unit is arranged to perform remote data gathering by:
controlling at least one said remotely controllable water flow control valve to place a respective said water outlet in fluid communication with the water inlet for a finite pre-set time period; and,
receiving said monitoring signal during said time period.

11. The emergency washing apparatus of claim 10, wherein the shower control unit is arranged to issue a warning signal if the measured rate of flow falls below a pre-set threshold water flow rate value.

12. The emergency washing apparatus of claim 11, wherein the shower control unit is arranged to repeat said remote data gathering if an interval of time since the most recent previous said remote data gathering exceeds a threshold time interval value.

13. An emergency washing apparatus adapted for generating a controlled spray or jet of water directed at a user for washing therewith, the emergency washing apparatus comprising:
a washing unit including a water inlet and one or more water outlets arranged for forming said spray or jet of water received from the water inlet;
one or more water flow control valves each operably coupled to a respective control switch arranged to operate the one or more water flow control valves to reversibly place a said water outlet in fluid communication with said water inlet;
one or more sensors configured to detect an operation of a said control switch and to transmit a monitoring signal indicating an occurrence of the operation;
the one or more sensors configured to sense one or more properties of the water received at the water inlet selected from: a water flow rate; a water pressure; a water temperature; and to transmit a respective measurement signal indicating a sensed property of the water;
a shower control unit which is located locally at, or remote from, the washing unit and which is arranged to receive said monitoring signals and said measurement signals and to issue a warning signal if said received monitoring signals and measurement signals collectively fail to comply concurrently with pre-set conditions thereby indicating a malfunction of the washing unit.

14. The emergency washing apparatus of claim 13, in which the one or more said control switches are each selected from: manually operable control switches.

15. The emergency washing apparatus of any of claim 14, in which concurrent compliance with said pre-set conditions is such that a condition applied to said received measurement signals is conditional upon said received monitoring signals.

16. The emergency washing apparatus of any of claim 15, in which the one or more sensors are configured to sense said water flow rate and the shower control unit is arranged to issue said warning signal if said measurement signal indicates a non-zero water flow rate and concurrently a said monitoring signal(s) indicates that no said water outlet is in fluid communication with said water inlet.

17. The emergency washing apparatus of any of claim 16, in which the one or more sensors are configured to sense said water flow rate and the shower control unit is arranged to issue said warning signal if said measurement signal indicates no water flow and concurrently a said monitoring signal(s) indicates that a said water outlet is in fluid communication with said water inlet.

18. The emergency washing apparatus of any of claim 17, comprising a first said water outlet and a second said water outlet, in which the one or more sensors are configured to sense said water flow rate and the shower control unit is arranged to issue said warning signal if said measurement signal indicates said water flow rate exceeding:
a first pre-set water flow rate threshold value and concurrently a said monitoring signal(s) indicates that only one of the first and second water outlets is in fluid communication with said water inlet; or,
a second pre-set water flow rate threshold value exceeding the first pre-set water flow rate threshold value and concurrently a said monitoring signal(s) indicates that both of the first and second water outlets is in fluid communication with said water inlet.

19. The emergency washing apparatus of claim 18, in which the first pre-set water flow rate threshold value is between 6 liters per minute and 12 liters per minute, and the second pre-set water flow rate threshold value is one of:
between 60 liters per minute and 100 liters per minute; between 30 liters per minute and 60 liters per minute; at least 100 liters per minute.

20. The emergency washing apparatus of any of claim 19, in which the one or more sensors are configured to sense said water pressure and the shower control unit is arranged to issue said warning signal if said measurement signal indicates said water pressure exceeding a pre-set upper pressure threshold value and concurrently a said monitoring signal(s) indicates that no said water outlet is in fluid communication with said water inlet.

21. The emergency washing apparatus of claim 20, in which the one or more sensors are configured to sense said water pressure and the shower control unit is arranged to issue said warning signal if said measurement signal indicates said water pressure less than a pre-set lower pressure threshold value, and concurrently a said monitoring signal(s) indicates that no said water outlet is in fluid communication with said water inlet.

22. An emergency washing apparatus adapted for generating a controlled spray or jet of water directed at a user for washing therewith, the emergency washing apparatus comprising:
a washing unit including a water inlet and one or more water outlets arranged for forming said spray or jet of water received from the water inlet;
a user identity apparatus configured to generate identification information for identifying the user of the washing unit and to transmit the user identification information;
a shower monitoring unit remote from the washing unit responsive to receipt of the transmitted user identification information to issue a telecommunications signal conveying:
information identifying a location of the washing unit; and, information identifying the user.

23. The emergency washing apparatus of claim 22, wherein the user identity apparatus comprises any one or more of:
a fingerprint detector configured for detecting a fingerprint of the user of the washing unit;
a camera configured for detecting the face of the user of the washing unit;
an RFID detector configured for detecting an RFID tag worn by the user of the washing unit.

24. The emergency washing apparatus of any of claim 23, in which the washing unit includes:
a manually operable switch mechanism configured for activating the formation of said spray or jet of water, and
the user identity apparatus includes said fingerprint detector is arranged upon the manually operable switch and configured detect a fingerprint of a user in contact therewith.

25. The emergency washing apparatus of any of claim 24, wherein the shower monitoring unit includes a data processor and a display unit configured to generate one or each of: geographical coordinates identifying the location of the washing unit; a map of an environment local to the washing unit upon which the location of the washing unit is shown, and to display the geographical coordinates, or the map, on a graphical user interface.

26. The emergency washing apparatus of any of claim 25, wherein the shower monitoring unit is configured to generate one or each of: geographical coordinates identifying the location of the washing unit; a map of an environment local to the washing unit upon which the location of the washing unit is shown, and to convey the geographical coordinates, or the map, within said information identifying the location of the washing unit in said telecommunications signal.

27. The emergency washing apparatus of any of claim 26, wherein the shower monitoring unit is configured to generate information selected from any one or more of: directions to the washing unit; medical data associated with the identity of the user, and to convey said information within said telecommunications signal.

28. An emergency washing apparatus adapted for generating a controlled spray or jet of water directed at a user for washing therewith, the emergency washing apparatus comprising:
a washing unit including a water inlet and one or more water outlets arranged for forming said spray or jet of water received from the water inlet;
a positioning unit configured to generate positioning information identifying a location of the washing unit and to transmit the positioning information;
a shower monitoring unit remote from the washing unit responsive to receipt of the transmitted positioning information to issue a telecommunications signal conveying information identifying the location of the washing unit.

29. The emergency washing apparatus of claim 28, including a geo-spatial positioning transceiver unit operably connected to a Global Navigation Satellite System (GNSS) or a terrestrial wireless telecommunications network, and configured to determine a position of the washing unit accordingly.

30. The emergency washing apparatus of any of claim 29, including user identity apparatus configured to generate identification information identifying the user of the washing unit and to transmit the user identification information;
wherein the shower monitoring unit is responsive to receipt of the transmitted user identification information to issue a telecommunications signal conveying:
information identifying the location of the washing unit; and, information identifying the user.

31. The emergency washing apparatus of claim 30, wherein the user identity apparatus comprises any one or more of:
a fingerprint detector configured for detecting a fingerprint of the user of the washing unit;
a camera configured for detecting the face of the user of the washing unit;
an RFID detector configured for detecting an RFID tag worn by the user of the washing unit.

32. The emergency washing apparatus of claim 31, in which the washing unit includes a manually operable switch mechanism configured for activating the formation of said spray or jet of water, and the user identity apparatus includes said fingerprint detector arranged upon the manually operable switch and configured detect a fingerprint of a user in contact therewith.

33. The emergency washing apparatus of claim 32, wherein the shower monitoring unit includes a data processor and a display unit configured to generate one or each of: geographical coordinates identifying the location of the washing unit; a map of an environment local to the washing unit upon which the location of the washing unit is shown, and to display the geographical coordinates, or the map, on a graphical user interface.

34. The emergency washing apparatus of claim 33, wherein the shower monitoring unit is configured to generate one or each of: geographical coordinates identifying the location of the washing unit; a map of an environment local to the washing unit upon which the location of the washing unit is shown, and to convey the geographical coordinates, or the map, within said information identifying the location of the washing unit in said telecommunications signal.

35. The emergency washing apparatus of any of claim 34, wherein the shower monitoring unit is configured to generate information selected from any one or more of: directions to the washing unit, medical data associated with the identity of the user, and to convey said information within said telecommunications signal.
